(19) United States
(12) Patent Application Publication
(10) Pub. No.: US 2005/0048444 A1
(43) Pub. Date: Mar. 3, 2005

(54) Teeth whitening composition and method

(76) Inventor: Alan A. Creamer, Carlsbad, CA (US)

Correspondence Address: John C. Serio;Brown Rudnick Berlack Israels LLP, One Financial Center, Boston, MA, 02111, US

(21) Appl. No.: 10/925,820

(22) Filed: Aug. 25, 2004

Related U.S. Application Data
(60) Provisional application No. 60/499,692, filed on Sep. 3, 2003.
(60) Provisional application No. 60/498,990, filed on Aug. 29, 2003.
(60) Provisional application No. 60/505,196, filed on Sep. 23, 2003.

(51) Int. Cl. A61C 3/00; A61K 7/20

(52) U.S. Cl. 433/215; 433/29; 424/53

(57) ABSTRACT

A dental whitening composition is formed from tablet formulation containing a metal ion catalyst and an alkaline pH raising compound and a peroxide solution having a concentration of about 1 percent to about 15 percent peroxide. The combination of the tablet formulation containing the metal ion within an alkaline composition along with the peroxide rinse forms a dental composition having foam like consistency, which whitens the surface of teeth. The tablet formulation can also be in the form of a gel or solution. The process of whitening teeth is accelerated by using a light emitting device producing a selected wavelength range. The light emitting device can be incorporated into a dental tray having a dental receiving area formed from a polymer having optical properties.

### CROSS REFERENCE TO RELATED APPLICATIONS

This patent application claims priority from U.S. Provisional Patent Application Ser. No. 60/499,692, filed on Sep. 3, 2003, U.S. Provisional Application Ser. No. 60/498,990, filed on Aug. 29, 2003 and U.S. Provisional Application Ser. No. 60/505,196, filed on Sep. 23, 2003, the contents of which are incorporated herein by reference in their entirety.

### FIELD OF THE INVENTION

The present invention relates to light activated dental compositions and a light emitting dental tray for treating teeth surfaces. In particular, the present invention is directed to dental compositions, such as tooth bleaching compositions, used in combination with a light emitting dental tray having reservoirs for holding the dental composition located adjacent the teeth surfaces to be treated.

### BACKGROUND OF THE INVENTION

Increasingly, it has become popular to whiten teeth that are naturally off white or have become stained by smoking or food intake. In order to whiten teeth in the past, people either have had them capped or have had veneers placed over them, which both entail costly and involved dental procedures. Recently, chemical bleaching of teeth has allowed people to whiten their teeth without resorting to these previously costly and involved dental procedures.

In early bleaching methods, dental patients who desired to have their teeth bleached had to utilize conventional dental office bleaching techniques. These techniques usually involved placing a peroxide solution on the teeth, protecting the sensitive soft tissues with a ligated rubber dam, and applying heat or light to the solution. Unfortunately, the lights used in dental office procedures are high power high voltage lights that desiccate teeth and cause greater postoperative sensitivity. Additionally, such dental office treatments are extremely time consuming as they typically required multiple appointments for a significant color change. The need for multiple professional office visits results in a procedure that is costly. Despite the expense and other problems associated with professional whitening of teeth, these professional methods are primarily concerned with the whitening of only the buccal, facial surface only.

Because of the time consuming nature of these multiple office visits and costs associated therewith, there has been a growing interest within the dental profession for in-home tooth bleaching products and methods. One of the first in-home approaches required a professional making an alginate impression of a patient's teeth; making a cast of the impression; vacuum forming a tray from the cast, and trimming the impression to exclude gingival coverage. In using this early home use apparatus and method, the patient is instructed to place several drops of bleaching solution into each area of the tray for each tooth to be bleached, placing the tray containing the bleaching solution in the mouth and expectorating any excess bleaching solution. Unfortunately, use of this apparatus and method requires the patient to change the bleaching solution every 1 to 2.5 hours, and remove the dental tray during meals. As a result, this in-home approach is expensive, time consuming, cumbersome and suffers from a lack of compliance.

As a result of this inconvenient and expensive in-home method, there have been more recent in-home approaches that embed various bleaching agents onto substrates such as fabric or fiber strips that a patient applies to their teeth at bedtime. One known in-home method uses various peroxides within gel foam as dental bleaching agents. The most commonly used dental bleaching agent is 10% carbamide peroxide (CO(NH₂)₂H₂O₂), also called urea hydrogen peroxide, hydrogen peroxide carbamide, and perhydrol-urea.

While useful results have been experienced using the foregoing in-home techniques, their effectiveness has been largely dependent upon such factors as type and intensity of tooth enamel stain, bleaching agent contact time, and the amount of available active ingredient in the bleaching agent. The advantage, however, is that the time commitment for the actual bleaching process takes place outside the dental office and without the need for professional application. Thus, the cost for these in-home procedures is substantially less than conventional in-office bleaching techniques.

Unfortunately, despite improvements in these in-home methods, there remain disadvantages and limitations to in-home bleaching products and techniques. A significant disadvantage of the known in-home approaches is the long application or contact time needed by these methods. Because of the required long contact time, bleaching agent must be frequently replaced or replenished during application. Replenishment is needed because of saliva dilution and swallowing of the bleaching agent causing the volume of bleaching agent in the dental application tray to diminish rapidly over time. Various studies have shown that after one hour, less than one-half the original volume of bleaching agent was present in an application tray. Thus, existing bleaching agents, because of their method of application and the need for a long contact time, need to be replenished about every hour in order to be effective.

Because of the inconvenience of replacing bleaching agents constantly and the long contact time needed, patient compliance is difficult to maintain and therefore in-home methods have not achieved the level of success as professional in-office bleaching techniques.

An additional problem with current in-home bleaching compositions and methods is that it often takes several weeks of application to see desired results. Although some methods have promoted lightening of teeth in shorter periods of time, noticeable results of in-home bleaching requires approximately 4 to 6 weeks. This lengthy period of treatment and compliance issues have resulted in dissatisfaction with many if not all of the present in-home compositions and methods.

It should therefore be appreciated that what is needed in the art are improved compositions and methods for faster and more efficient treatment of tooth surfaces which facilitate greater patient compliance and satisfaction. Additionally, it would be a significant advancement in the art to provide fast acting dental bleaching compositions for treating tooth surfaces which do not need to be continuously replaced so that patient compliance is enhanced.

Thus there is a need for a tooth whitening method that will bleach stained teeth and inhibit tooth yellowing that does not require complex, time consuming and expensive equipment.

### SUMMARY OF INVENTION

In a first illustrative embodiment, according to the invention, a tablet formulation containing a metal ion catalyst and an alkaline pH raising compound is chewed by a patient followed by rinsing their teeth with a peroxide composition having a concentration of about 1 percent to about 15 percent peroxide. The combination of the tablet formulation containing the metal ion within an alkaline composition along with the peroxide rinse forms a dental composition having foam like consistency, which whitens substantially all of the surfaces of the patient's teeth. The process of whitening the patient's teeth is accelerated by using a light-emitting device producing a selected wavelength range to assist the decomposition of the peroxide intra orally by activating the metal ion catalyst within the dental composition. The light emitting device can be incorporated into a dental tray that captures the whitening composition and holds the whitening composition on the surface of the teeth.

In a further illustrative embodiment, according to the invention, a solution having a gel consistency comprising selected peroxides and selected transitional metal ions, such as ionized silver, zinc, manganese or the like is sprayed onto the surface of a patient's teeth. The sprayed solution is followed by a rinse of an additional low concentration peroxide, such as hydrogen period or carbamide peroxide, or calcium peroxide. The additional peroxide can be applied to the teeth using a conventional dental tray or by merely rinsing the oral cavity with a low concentration solution. The dental tray can further include a light source having a selected wavelength that activates the metal ion within the dental composition. The pH of the above spray is adjusted to about 6 to about 8 or above by the use of an alkaline agent, which allows for a faster decomposition of the peroxide. The light source is directed within the oral cavity for a period of about two minutes to about 20 minutes. The selected light source activates the photo sensitive metal ions and further produces heat hastening the decomposition of peroxides thereby accelerating the whitening effect.

It is contemplated within the scope of the invention that the light source may be embedded into a conventional dental tray, where the dental tray further allows the containment of the above dental composition allowing the patient to hold the whitening composition within the mouth for a desired period of time.

The light source in one illustrative embodiment uses light emitting diodes or traditional small bulbs that are either blue, cyan, amber or white in color. The temperature produced by such a bulb or LED illumination raises the temperature of the dental composition. The increased temperature helps decompose the peroxides by a factor of about 2.4 for every 10° C. rise in temperature. The light source according to the invention is battery powered allowing hands free operation of the light equipped dental tray. The lighting system according to the invention is a low voltage, low intensity system that works well because of proximity of the treating surfaces to the light source itself. It is envisioned that other power sources may be employed, such as, for example, A/C wall outlet, etc.

In a further illustrative embodiment the dental try is fabricated from a light transmitting polymer that acts as a fiber optic transmitter allowing the light source to be emitted from substantially all surfaces of the dental tray whitening front, sides and the back of teeth.

### BRIEF DESCRIPTION OF THE DRAWINGS

The foregoing and other features and advantages of the present invention will be more fully understood from the following detailed description of illustrative embodiments, taken in conjunction with the accompanying drawings in which:

FIG. 1 is a flow diagram depicting an illustrative method according to the invention;

FIG. 2 is a flow diagram depicting an alternative illustrative method according to the invention;

FIG. 3 is a top view of a dental tray according to the invention; and

FIG. 4 is a side perspective view of the dental tray, with parts separated, according to the invention.

### DETAILED DESCRIPTION OF THE INVENTION

Detailed embodiments of the present invention are disclosed herein, however, it is to be understood that the disclosed embodiments are merely exemplary of the invention, which may be embodied in various forms. Therefore, specific functional details disclosed herein are not to be interpreted as limiting, but merely as a basis for the claims and as a representative basis for teaching one skilled in the art to variously employ the present invention in virtually any appropriately detailed embodiment.

According to the invention, a dental whitening composition having teeth whitening properties is disclosed. In a first illustrative embodiment, the dental whitening composition is formed from a first gel and a second gel. The first gel, according to the invention, is a peroxide gel having about 1 to about 15 percent peroxide by weight. The peroxide gel composition, according to the invention, is formed by mixing approximately 3 gm of a gelling powder, Micropore Gel® powder, Bioserve, San Diego, Calif., with approximately 97 gm of deionized water. The gelling powder is mixed with the deionized water for approximately one-half hour until a clear flowable gel is formed.

The flowable gel is then mixed with approximately 10.98 gm of a 50 percent hydrogen peroxide solution and stirred slowly for approximately 15 minutes. In this first illustrative embodiment Peralkali®, a 50% peroxide solution, Degussa Manufacturing, was used. It is contemplated that other peroxides known in the art may be used such as carbamide peroxide, potassium peroxide, calcium peroxide, or the like. A citric acid buffer is added until the mixture achieves a pH of approximately 3.5. Approximately 2 gm of a dry flavoring is added to the buffered gel composition. The formed buffered peroxide gel will stabilized to about a pH of 5 after about 24 hours.

The second gel, is an accelerating gel formed by mixing approximately 3 gm of Micropore® Gel powder with approximately 97 gm of deionized water. This mixture is stirred for approximately one-half hour until a clear flowable gel is formed. The flowable gel is mixed with approximately 3.2 gm of silver ion solution (500 ppm). Approximately 10.40 gm of Tri (hydroxymethyl)-aminomethane, Angus Chemical Company, Buffalo Grove, Ill., is added to approximately 1 gm of water forming a buffering solution. The buffering solution is added to the ionized gel solution forming the accelerating gel. The accelerating gel is contained within an opaque container until use with the peroxide gel.

The whitening composition is formed by applying the peroxide gel to the inventive dental tray along with the accelerating gel. The combined gels form a whitening composition that is activated by a selected light source within the inventive dental tray.

In a further illustrative embodiment, a metal ion accelerator is provided in a tablet formulation having a metal ion catalyst and an alkaline component. After apply a peroxide gel composition according to the invention, the accelerator tablet is chewed by a patient. Transitional metal ions such as ionized silver, zinc, manganese or the like may be used as a catalyst according to the invention. It is contemplated that other ions that are photo sensitive and strongly reactive to light may be used. Alkaline compounds such as sodium bicarbonate, sodium hydroxide, [tri(dydroxymethyl)aminomethane] or the like may be used to raise the pH of the whitening composition. In this further illustrative embodiment the tablets are comprised of deionized water approximately 75% by weight; ionized silver ion approximately 21% by weight; Tris Amine approximately 4% by weight; and flavoring.

In yet a further illustrative embodiment a peroxide solution rather than a gel can be used as an oral rinse containing about 1 percent to about 15 percent hydrogen peroxide. It is contemplated that other peroxides known in the art may be used such as carbamide peroxide, potassium peroxide, calcium peroxide, or the like. The combination of the metal ion alkaline tablet with the peroxide rinse forms a dental composition within a patient's oral cavity having foam like consistency. This dental composition whitens the surface of the patient's teeth.

According to a further illustrative embodiment, an activated dental whitening composition can be formed from two solutions. The first solution contains approximately between 1 percent and 15 percent hydrogen peroxide with selected flavoring. It is contemplated within the scope of the invention that the first solution can also contain colorants such as pigments and dyes to impart a desired color to the solution.

The second solution contains an activating silver ion solution having approximately 10 ppm to 1000 ppm of silver ion. In an illustrative embodiment the second solution contains approximately 125 ppm of silver ion in about 100 gm of deionized water. The second solution is buffered by adding approximately 10.32 grams of Tri(hydroxymethyl)aminomethane added to about 1 gm of water forming a buffering solution having a pH of about 10. It is contemplated within the scope of the invention that a buffers such as Tri(hydroxymethyl)aminomethane can be used in a concentration in the second solution of about 1 percent by weight to about 15 percent by weight. This buffering solution is added to the silver ion solution along with approximately 2 gm of dry flavoring. According to the invention the first solution containing the peroxide and the second solution containing the silver ion are applied to the patient's teeth by spraying each solution in a predetermined amount onto the patient's teeth. It is contemplated within the scope of the invention that these solutions can be used alone or in conjugation with the peroxide gel and accelerator gel according to the invention. It is further contemplated that these solutions and gels can be used along with the accelerator tablets according to the invention.

A light source having a wavelength selected that is specific to the photo-sensitive metal ion within the dental whitening composition may be used to activate the dental whitening composition by increasing the decomposition of the peroxide used. This decomposition allows for a hastening of the whitening effect and a dramatic decrease in contact time. According to the invention, a light bulb or LED producing wavelengths forming blue, cyan, amber or white light can be used to activate the photo ions. It is contemplated, within the scope of the invention, that the desired selected light wavelength can be produced by a bulb or LED selected or by the use of an optical wavelength filter allowing for the selection of a desired wavelength range.

In a first illustrative embodiment, a LED, Nichia Corporation, of Japan, part number, NSPB 310a, is employed producing a wavelength within the range of about 430 nm to about 490 nm. The LED used in this illustrative embodiment has the following specifications: Chromaticity Coordinate Typical (x,y) 0.130 to 0.75; Luminous intensity (mcd) 3900; Forward Voltage 3.5 max 4.0; Direction Characteristics 30 degrees Size 3.0 mm.

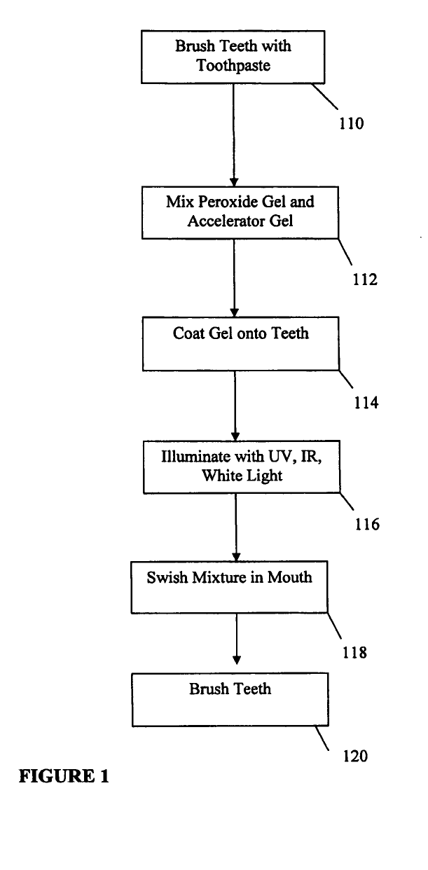

Referring to FIG. 1, the dental whitening composition is employed in a teeth whitening procedure that includes brushing a patient's teeth (step 110). The dental whitening composition is formed from a peroxide gel and an accelerator gel forming an activated gelling agent (step 112). The activated gelling agent is then coated onto the teeth and added to a dental tray's dental receiving area before the patient inserts the tray into their mouth and illuminates the surfaces of their teeth with the light source embedded into the Dental tray. (step 114). The light source illuminates the coated teeth for approximately two minutes (step 116). The activated gelling agent mixture is swished about the patient's mouth after illumination (step 118). The patient's teeth are brushed (step 120).

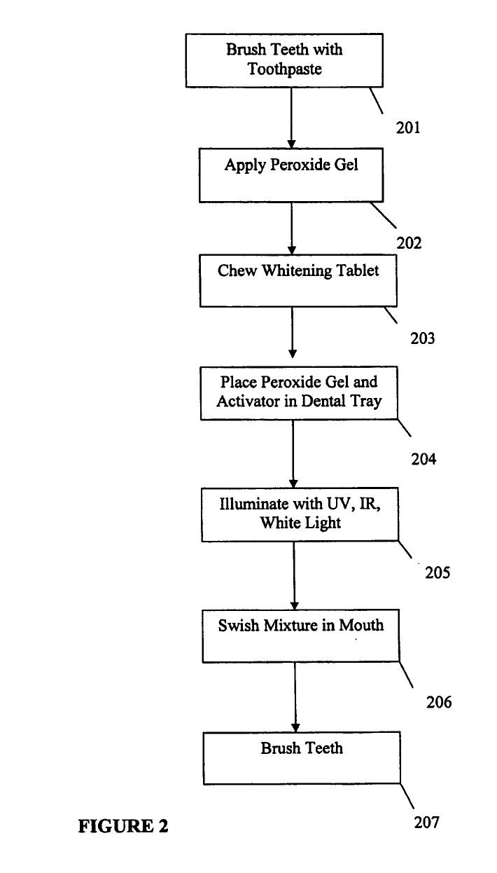

Referring to FIG. 2, the dental whitening composition is employed in a teeth whitening procedure that includes brushing the patient's teeth (step 201) and placing a peroxide gel onto the patient's teeth surfaces (step 202). The patient then chews an activator tablet (step 203) forming a whitening composition. The patient then places peroxide gel into the dental receiving area along with activator gel (step 204), which is placed on top of the peroxide gel. The resulting whitening composition is illuminated for approximately two minutes or more (step 205). The illuminated composition is then swished around the oral cavity for as long as desired (step 206). The oral cavity is rinsed and the patient brushes their teeth (step 207).

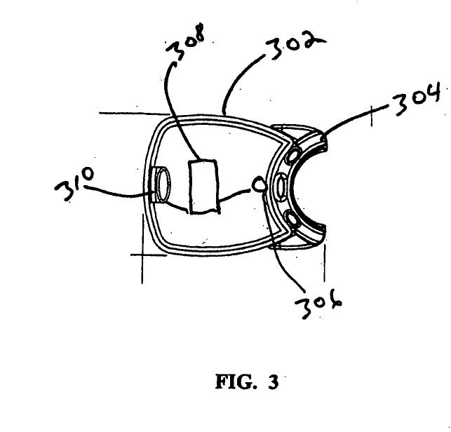

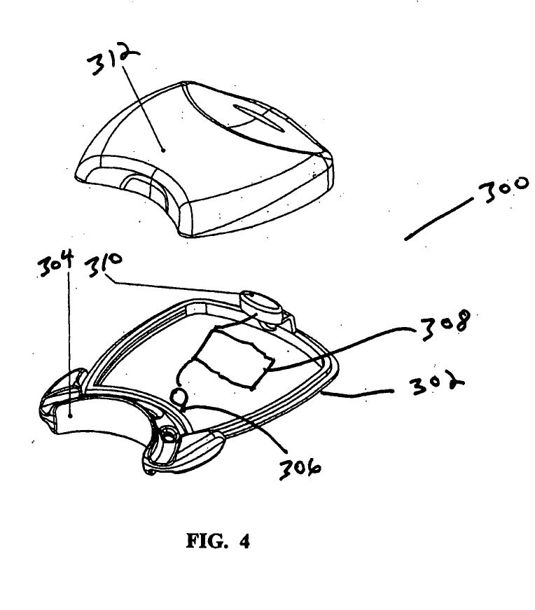

According to the invention, the light source can be embedded in a dental tray 300 or be in optical communication with the dental tray 300. As shown in FIG. 3 and FIG. 4, the dental tray 300 has a bottom rigid tray 302 having a dental receiving area 304. The dental receiving area 304 is formed in the shape of orthodontic wire and the curvature of a patient's teeth. The dental receiving area 304 is loose fitting allowing both sides of the teeth to receive gel or rinse material.

The bottom rigid tray 302 is formed from a rigid polymeric material. The dental receiving area 304 is formed from a pliable polymeric material and is fixably attached to the bottom rigid tray 302. In a first illustrative embodiment, the dental receiving area 304 is formed from a pliable silicone that is transparent to the wavelength range of a light source attached to the bottom rigid tray 302. It is contemplated within the scope of the invention that the dental receiving area 304 can be fabricated from any polymeric compound that is pliable and translucent to a selected wavelength range. It is further contemplated within the scope of the invention that the polymeric composition forming the dental receiving area 304 has light transmitting properties allowing the dental receiving area 304 to act as a fiber optic bundle transmitting light to all areas of the dental receiving area 304.

The bottom tray 302 in this first illustrative embodiment is equipped with at least one LED 306 that produces a light having a selected wavelength. In a first illustrative embodiment several LED 306 are mounted in the bottom tray 302 so that their emitted light is directed around the curvature of the dental receiving area 304. It is contemplated within the scope of the invention that focusing optics or filtering optics can be mounted in front of the light emitting portion of the LEDs 306. These optics can direct the emitted light or filter the emitted light to a desired wavelength range. It is also contemplated within the scope of the invention that a singular LED or light source can be positioned in optical communication with a fiber optic bundle that delivers the emitted light from the light source to desired locations-within the dental receiving area 304.

The LEDs 306 are powered by a battery pack 308 that is in electrical communication with each LED 306. The battery pack 308 in a first illustrative embodiment is a standard 9 volt battery. It is contemplated within the scope of the invention that a rechargeable battery or batteries may be used that produce the needed electrical power specifications for the LED 306 or other light source used. It is also contemplated that the LEDs 306 can be powered by standard household electricity using a transformer capable of providing the desired voltage or recharging rechargeable batteries. The electrical communication of the LEDs 306 is controlled by a single pole electrical switch 310 allowing a user to power on and off the LEDs 306. In a first illustrative embodiment an electrical resistant element (not shown) is incorporated into the electrical circuit allowing the dental tray to warm to approximately 100° Fahrenheit. It is contemplated within the scope of the invention that the electrical switch 310 may further contain a timer element allowing the user to select a desired operational time and in one illustrative embodiment a pre-selected operational time.

The battery pack 308, the electrical communication to the LED 306 and the electrical switch 310 are protected from dental compositions utilized and outside elements by the use of a cover plate 312 that is removably attached to the bottom plate 302. The cover plate 312 is formed from a polymeric material such as ABS, polycarbonate, or the like. It is contemplated within the scope of the invention that the bottom plate 302 and the cover plate 312 can be fabricated from various metals.

Although the illustrative embodiments show the use of metal ions within the activating solution, gel or tablet, it will be understood by those skilled in the art that ions other than metal ions may be used to accelerate the breakdown of peroxides during the bleaching process. Likewise it will also be understood that ions that are highly sensitive to heat may be used to accelerate the breakdown of peroxides during the bleaching process.

The principles, preferred embodiments and modes of operation of the presently disclosed light activated dental whitening composition and light embedded tray have been described in the foregoing specification. The presently disclosed light activated dental whitening composition and light embedded tray, however, is not to be construed as limited to the particular embodiments shown, as these embodiments are regarded as illustrious rather than restrictive. Moreover, variations and changes may be made by those skilled in the art without departing from the spirit and scope of the light activated dental whitening composition and light embedded tray and disclosed herein and recited in the appended claims.

1. A method for whitening teeth comprising the steps of:
providing an alkaline composition having a photo-sensitive ion;
providing a bleaching agent;
mixing the alkaline composition with the bleaching agent forming a whitening composition applying said whitening composition to teeth; and
irradiating the whitening composition with a selected wavelength range that excites said photo-sensitive ion.

2. The method according to claim 1, wherein said photo-sensitive ion is a metal-ion selected from the group consisting of ionized silver, ionized zinc and ionized manganese.

3. The method according to claim 1, wherein said alkaline composition is formed from an alkaline compound selected from the group consisting of sodium bicarbonate, sodium hydroxide and [tri(dydroxymethyl)aminomethane].

4. The method according to claim 1, wherein said bleaching agent is a peroxide.

5. The method according to claim 4, wherein said peroxide is selected from the group consisting of carbamide peroxide, calcium peroxide, potassium peroxide and hydrogen peroxide.

6. The method of claim 1, further comprising the step of heating said whitening composition.

7. The method according to claim 1, wherein said whitening composition is a viscous foam that allows greater contact time with said teeth.

8. The method according to claim 1, wherein said excited photo-sensitive ion decreases the time needed for decomposition of said bleaching agent.

9. The method according to claim 1, wherein said selected wavelength range is from about 430 nm to about 490 nm.

10. The method according to claim 1, wherein said selected wavelength range is produced by the emittance of a light source.

11. The method according to claim 1, wherein said selected wavelength range is produced by the filtered emittance of a light source.

12. The method according to claim 10 wherein said light source is a LED.

13. The method of claim 1, wherein said selected wavelength is delivered to all exposed surfaces of said teeth.

14. An apparatus for whitening teeth comprising:
a bottom plate and a cover plate;
a dental receiving area fixably attached to said bottom plate; and
a light emitting device incorporated into said dental receiving area, said light emitting device producing a selected wavelength range.

15. The dental whitening apparatus according to claim 14, wherein said light emitting device comprises at least one LED.

16. The dental whitening apparatus according to claim 15, wherein said at least one LED is in optical communication with said dental receiving area.

17. The dental whitening apparatus according to claim 15, wherein said at least one LED emits light in a selected wavelength range of about 430 nm to about 490 nm.

18. The dental whitening apparatus according to claim 15, wherein said dental receiving area is fabricated from a light transmitting composition.

19. The dental whitening apparatus according to claim 14, wherein said light emitting device is in optical communication with a fiber optic bundle said fiber optic bundle directing light to at least one portion of said dental receiving area.

20. The dental whitening apparatus according to claim 14, wherein said selected wavelength range is produced by the emittance of a light source.

21. The dental whitening apparatus according to claim 14, wherein said selected wavelength range is produced by the filtered emittance of a light source.

22. The dental whitening apparatus according to claim 14, wherein said selected wavelength is delivered to all exposed surfaces of said teeth.
